(19) United States
(12) Patent Application Publication
(10) Pub. No.: US 2012/0253359 A1
(43) Pub. Date: Oct. 4, 2012

(54) SYSTEMS AND METHODS FOR LEAD PLACEMENT OPTIMIZATION DURING LEAD IMPLANTATION

(75) Inventors: Steve Koh, South Pasadena, CA (US); Gene A. Bornzin, Simi Valley, CA (US)

(73) Assignee: PACESETTER, INC., Sylmar, CA (US)

(21) Appl. No.: 13/076,104

(22) Filed: Mar. 30, 2011

(51) Int. Cl. A61N 1/05; A61B 19/00; A61N 1/36

(52) U.S. Cl. 606/129; 607/116; 607/72

(57) ABSTRACT

Disclosed herein is a method of optimizing the implantation of an implantable medical lead into a patient to optimize electrotherapy administered via the lead. The method includes: inserting the lead into the patient, the lead including a first electrode; providing a second electrode in the patient, wherein the second electrode is not part of the lead; generating an electrical vector between the first electrode and second electrode, the electrical vector being generated as the lead is being implanted; analyzing the electrical vector as the lead is being implanted; and optimizing the implantation of the lead based off of the analysis of the electrical vector to optimize electrotherapy administered via the lead.

### FIELD OF THE INVENTION

The present invention relates to medical devices and methods. More specifically, the present invention relates systems and methods for optimizing the placement of implantable medical leads.

### BACKGROUND OF THE INVENTION

Pacemakers and implantable cardioverter defibrillators (“ICD”) (i.e., pulse generators) are used to provide electrotherapy to a patient's heart via leads extending from the pulse generator to heart tissue of a lead implantation site. To optimize the administration of the electrotherapy, lead placement must be optimized.

There is a need in the art for systems, devices and methods for optimizing lead placement during lead implantation.

### BRIEF SUMMARY OF THE INVENTION

Disclosed herein is a method of optimizing the implantation of an implantable medical lead into a patient to optimize electrotherapy administered via the lead. In one embodiment, the method includes: inserting the lead into the patient, the lead including a first electrode; providing a second electrode in the patient, wherein the second electrode is not part of the lead; generating an electrical vector (e.g., constant current) between the first electrode and second electrode, the electrical vector being generated as the lead is being implanted; analyzing the electrical vector (e.g., voltage as a surrogate of impedance) as the lead is being implanted; and optimizing the implantation of the lead based off of the analysis of the electrical vector to optimize electrotherapy administered via the lead.

Also disclosed herein is a method of employing extra-cardiac impedance to intra-operatively optimize lead placement. In one embodiment, the method includes: during the implantation of a lead, generating an electrical vector between a lead electrode supported on the lead and another electrode not supported on the lead but at least partially positioned within a patient; during the implantation of the lead, analyzing the electrical vector; and guiding the implantation of the lead based at least in part off of the analysis of the electrical vector.

Further disclosed herein is a delivery tool for implanting a lead into a patient. In one embodiment, the tool includes a tubular body, a first electrode, and a conductor. The tubular body includes a proximal end, a distal end, and a lumen extending longitudinally through the tubular body between the proximal end and the distal end. The first electrode is supported on the tubular body near the proximal end in such a manner that the first electrode can be displaced longitudinally along the tubular body. The conductor extends proximally from the electrode. In one version of this embodiment, the tubular body is part of an introducer sheath or a catheter. In one version of this embodiment, the tool also includes a system coupled to a proximal end of the conductor and configured to analyze an electrical vector generated between the first electrode and an electrode of a lead delivered via the tool. The electrode of the lead may be positioned in a SVC of a patient. The system may analyze the electrical vector with respect to extra-cardiac impedance.

Also disclosed herein is a method of optimizing an implantation of an implantable medical lead. In one embodiment, the method includes: identifying a characteristic of extra cardiac impedance; and employing the characteristic as a surrogate of cardiac output, wherein the characteristic is monitored during lead implantation so as to position the lead to optimize cardiac output. The characteristic may include at least one of Zarea, slope, max, min, or peak-to-peak. The method may also include optimizing a pulse generator parameter to optimize cardiac output by monitoring the characteristic. The pulse generator parameter may include at least one of AVD, V-V timing, lead configuration, or pacing mode.

While multiple embodiments are disclosed, still other embodiments of the present invention will become apparent to those skilled in the art from the following detailed description, which shows and describes illustrative embodiments of the invention. As will be realized, the invention is capable of modifications in various aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1A is a graphical depiction of CO versus Z Area.

FIG. 1B is a graphical depiction of peak-to-peak impedance versus AVD.

FIG. 1C is a graphical depiction of Z versus Surface ECG.

FIG. 1D is a graphical depiction of dZ/dt versus Ao BP LVP.

FIG. 2 is a flow chart of a first method of employing ECI in the implantation of leads.

FIG. 3A is a diagram of a patient when the first method depicted in FIG. 1 is employed.

FIG. 3B is a diagram of a patient wherein another embodiment of the first method is employed,

FIG. 4 is a flow chart of a second method of employing ECI in the implantation of leads.

FIG. 5 is a diagram of a patient when the second method depicted in FIG. 4 is employed.

FIG. 6 is an isometric view of an electrode-equipped introducer and the PSA-programmer for use therewith, the introducer configured for use in a third method.

FIG. 7 is a flow chart of the third method of employing ECI in the implantation of leads.

FIG. 8 is a diagram of a patient when the third method depicted in FIG. 7 is employed.

### DETAILED DESCRIPTION

Disclosed herein are systems and methods for determining electrical characteristics during lead implantation to optimize lead placement at the lead implantation site to optimize the electrotherapy to be administered to the heart via the leads. For example, electrical vectors (e.g., constant current) may be generated between various electrodes or coils of the lead and the case of the pulse generator occupying a pocket formed in the patient's chest, temporary electrodes occupying the same type of pocket, or electrodes mounted on an introducer sheath. These electrical vectors can then be analyzed during lead implantation to determine the optimal locations for the implantation of the lead in the patient's cardiovascular system. For example, in analyzing the electrical vectors, voltage may be used as a surrogate of impedance.

Various electrical characteristics may be analyzed during lead implantation to optimize lead placement so as to optimize the electrotherapy to be administered to a patient's heart. For example, extra-cardiac impedance (“ECI”), which is measured between the superior vena cava (“SVC”) coil and the case (i.e., SVC-to-Case) of the pacemaker or ICD (“pulse generator”), provides an impedance signal with features that yield excellent correlation with LV contractility (“LVdp/dt”), cardiac output (“CO”), and systolic blood pressure.

Lead placement may be intra-operatively optimized by computing hemodynamic improvement while performing cardiac resynchronization therapy (“CRT”) to atrial pacing using an identical pacing rate and using a pacing system analyzer (“PSA”) with bi-ventricular (“BiV”) pacing capability. If CRT does not provide acute hemodynamic improvement, the leads may be repositioned or, in the case where a quad-pole left ventricular (“LV”) lead is placed, a different electrode may be selected.

SVC-to-Case impedance measured intra-operatively may be used to optimize lead placement for both LV leads and right ventricular (“RV”) leads. Lead placement optimization can be a mechanism for improving CRT performance in patients and improving the non-responder rate, thereby increasing the number of patients who could potentially benefit from CRT. ECI measurement capability may be provided with a PSA that has been combined with a programmer, such as, for example, the MERLIN™ programmer as marketed by St. Jude Medical, Inc.

As can be understood from Table A provided immediately below, upon collection of an impedance waveform, parameter features such as area, slope, max, min, peak-to-peak, etc. may be used as a surrogate for cardiac output. Based on these parameters, which give max CO, pacemaker parameters such as AVD, V-V timing, lead configuration, and pacing mode may be determined intraoperatively at bed side, at follow-up, or even automatically.

TABLE A

| Parameters | Description |
|---|---|
| minZ | min of Z |
| maxZ | max of Z |
| minZtime | time of minZ |
| mindZ_dt | min amplitude of Ensemble_dZ_dt |
| EjectStartTime | time of min of dZ/dt |
| EjectEndTime | time of max of dZ/dt |
| ejection_time | EjectEndTime − EjectStartTime |
| mintime | time of min of Ensemble_dZ_dt |
| DeltaZ | maxZ − minZ |
| Zarea | area under EnsembleZ from EjectStartTime to minZtime |

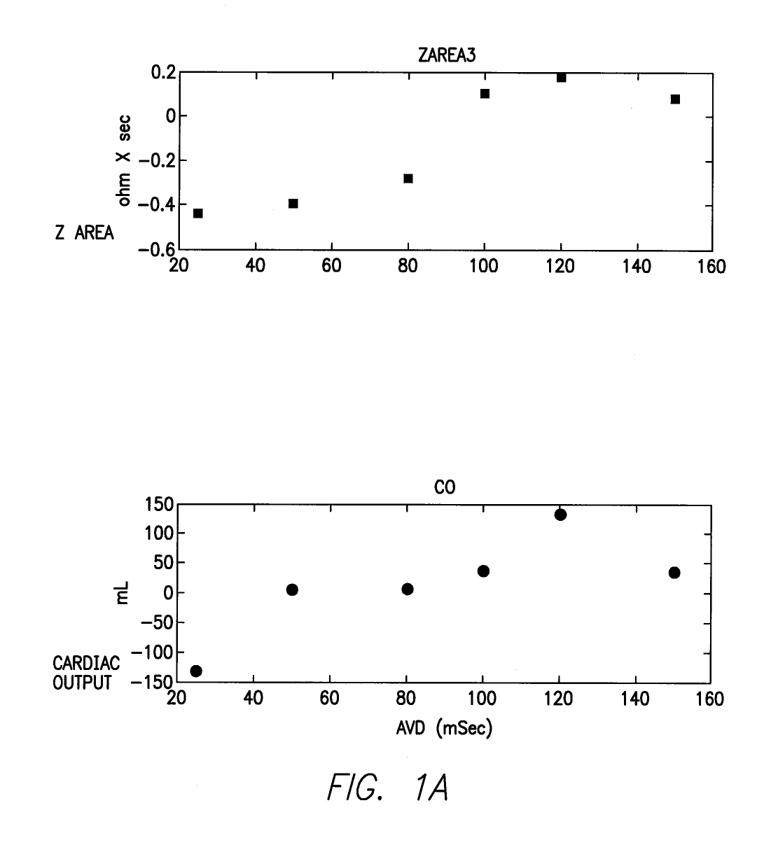

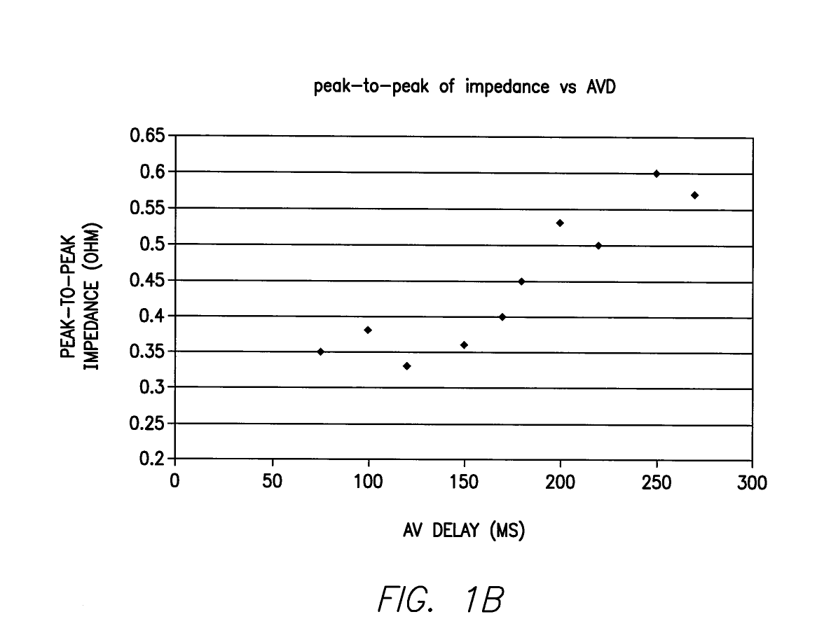

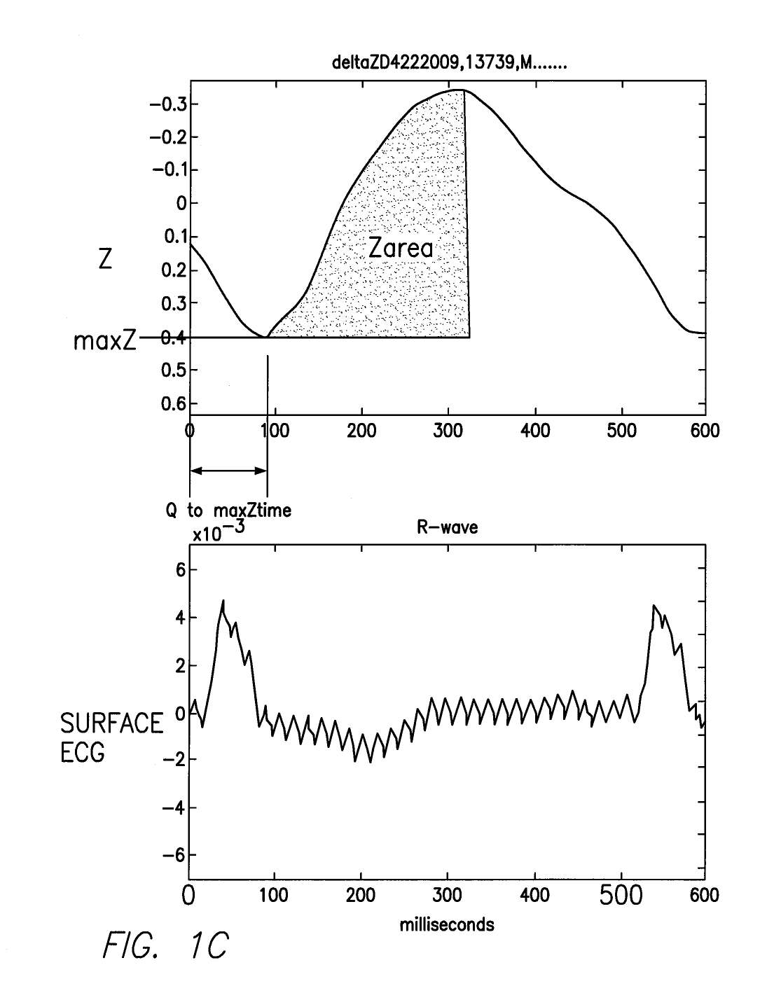

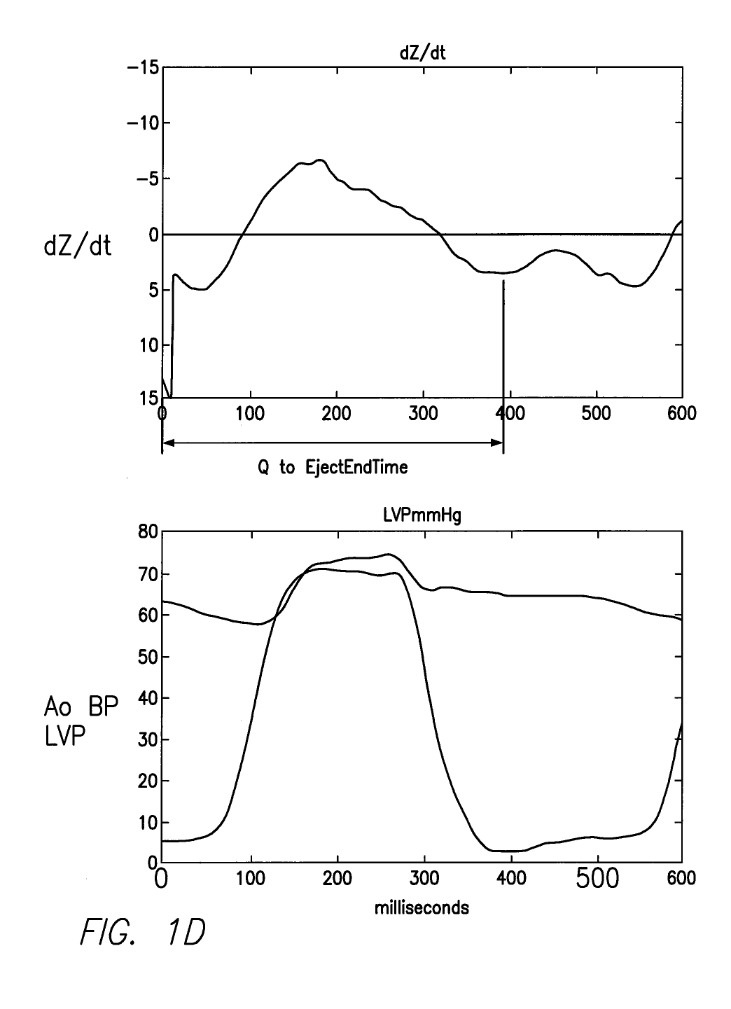

For example, as can be understood from the graphical depiction of CO versus Z Area in FIG. 1A, the analysis of the SVC-to-Case vector can provide high quality hemodynamic information. In this case, the hemodynamic performance information is not related to optimal lead location but rather atrial-ventricular (“AV”) delay optimization. Z area is the area measured underneath the SVC-to-Case impedance-gram. In generating the data depicted in FIG. 1A, the AV delay was varied between 25 ms and 150 ms, while performing simultaneous Bi-V pacing.

As can be understood from FIG. 1A, the greatest CO as measured by the Fick Method occurs at 120 ms AV delay. The Z area parameter from the ECI measurement shows the maximum area at 120 ms AV delay. Furthermore, the general upside down U-shape of the AV delay versus CO and AV delay versus Z area are very similar and show excellent concordance. Thus, as can be understood from FIG. 1A, ECI can be analyzed for intra-operative assessment of CRT efficacy.

Where there is an increase in AVD, there will also be an increase in a parameter derived from impedance. For example, as indicated in FIG. 1B, peak-to-peak impedance (also known as delayZ) is a good surrogate for CO. Specifically, FIG. 1B teaches as AVD increases, hemodynamic measured by extra-cardiac impedance measurement improved (in this specific plot); this is reflected as peak-to-peak amplitude (also called DeltaZ in table A) since minimum of Z (valley) is smaller and max of Z is going higher. Smaller Z (negative) valley value means more fluid is moving thru the Aortic arch during systolic and “dried up” in Diastolic phase, which allow next cycle of cardiac pump to work more efficiently with less resistance. The specific plot in FIG. 1B showed that maximum CO happened near 250 ms of AVD and as AVD increased from 100 ms to 250 ms, CO improved and peaked out at 250 ms.

As can be understood from FIGS. 1C and 1D, Zarea, Q to EjectionEndtime, and Q to minZtime were found to be good surrogates for SVC-to-Case impedance signals. For example, at 300 ms, Z peaks up (goes to min) due to max blood flow to aortic arches and slowly increase as the aortic arches dry up. More specifically, FIG. 1C teaches the area under the Z signal from time of maxZ to time of minZ can be used as surrogate of CO. These maxZ and minZ are very easy to detect rather than a full cardiac cycle of Z. The advantage of Z area is that it reflects both time and Z amplitude factor. As a result, Z area is a better index of blood volume rather than instantaneous blood flow. It should be noted that, to better reflect a traditional blood pattern, the y-axis is reversed (negative being top and positive being bottom) in FIG. 1C.

FIG. 1D teaches another interpretation of Z using a differentiator. For example, as shown in the upper or first plot of FIG. 1D, by choosing a min point after a zero crossing, which is called EjectEndTime, Q-to-EjectEndTime can be determined. If AVD or another programmable parameter is optimal, Q-to-EjectEndTime should be the shortest. The lower or second plot of FIG. 1D shows corresponding of AoBP and LVP. For example, LVP clearly showed that near 380 ms, LVP ejection is completed and this hemodynamic change can also be detected from dZ/dt as Q-to-EjectEndTime.

It should be noted that for the plots in the various figures, the mean was subtracted from raw waveform. Consequently, the plots have a mean of zero.

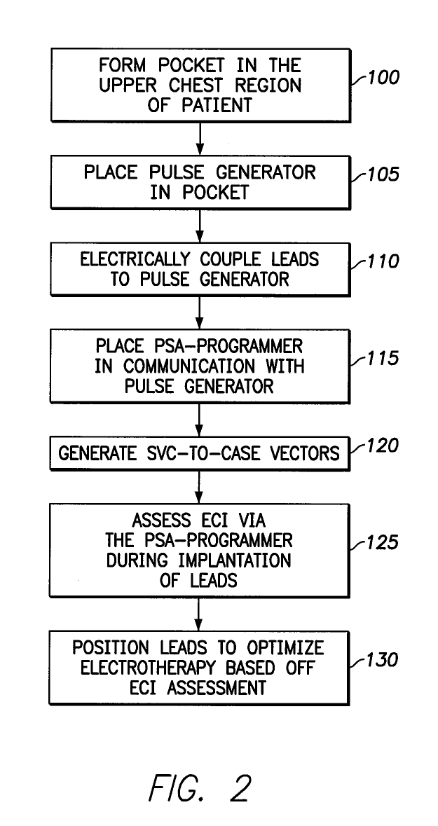

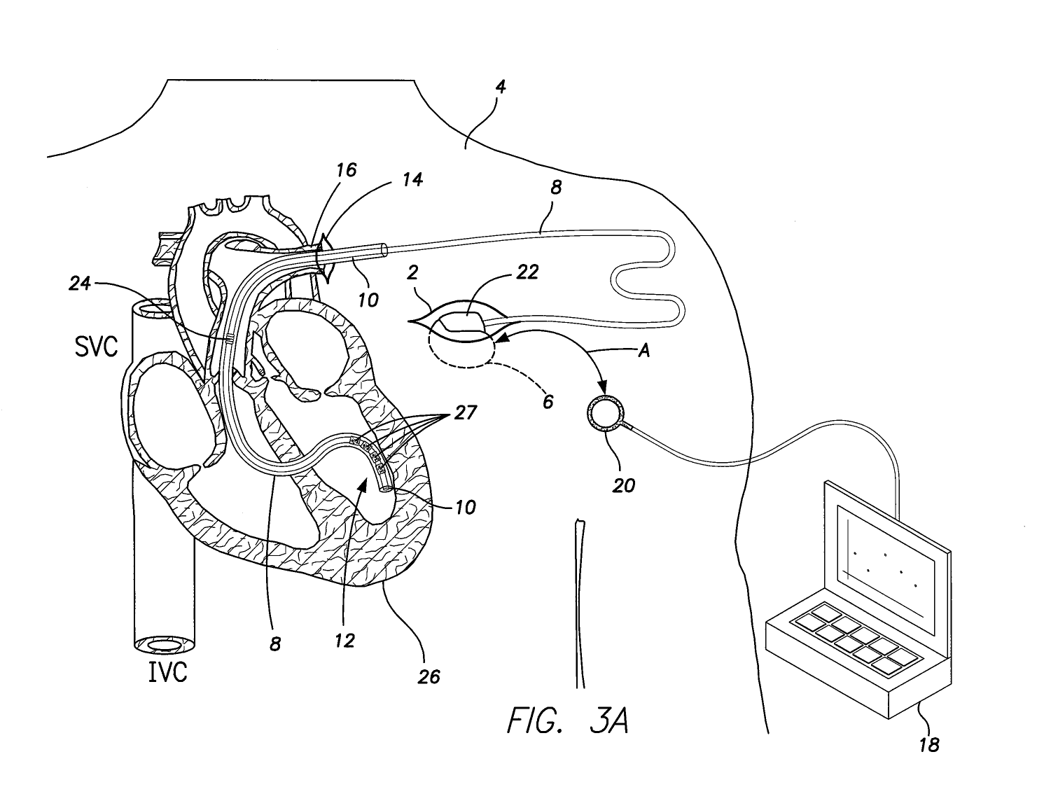

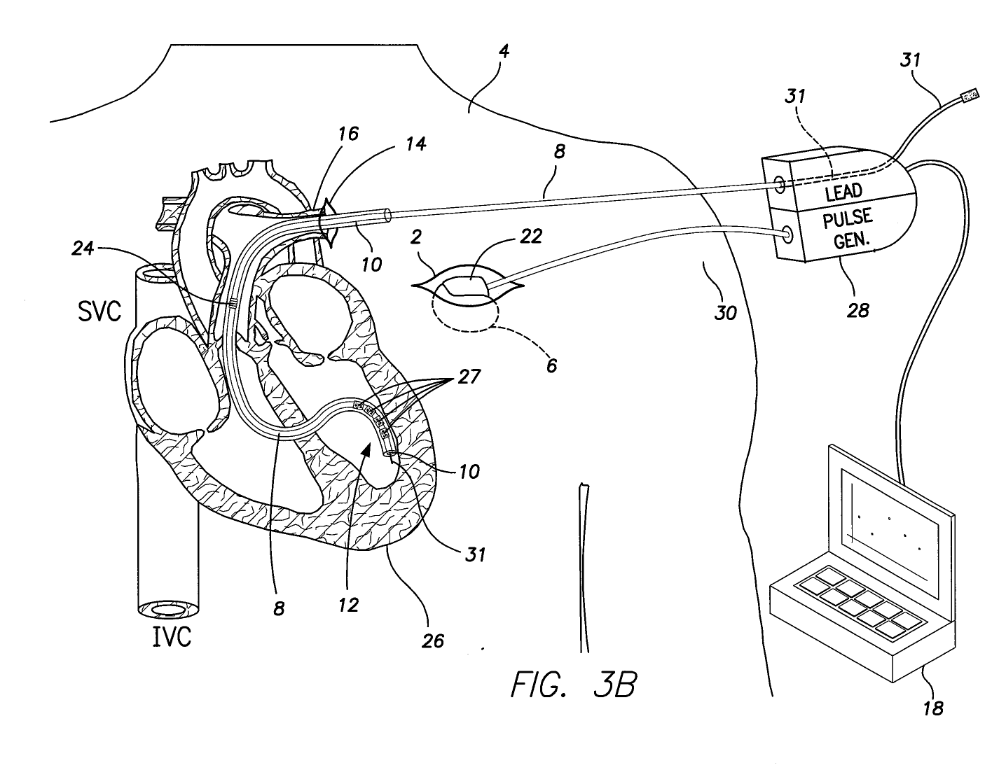

For a discussion of a first method of employing ECI analysis intra-operatively to optimize lead placement, reference is made to FIGS. 2 and 3A, which are, respectively, a flow chart and diagrams of a patient wherein the first method is employed. As can be understood from FIGS. 2 and 3A, in one embodiment, a pocket 2 is formed in the upper chest region of the patient 4 [block 100]. The pulse generator 6 is placed in the pocket [block 105]. The lead(s) 8 to be implanted extend into the patient's cardiovascular system via an introducer tubular body 10 (e.g., sheath, catheter, etc.) extending to the implant location 12 through a percutaneous access 14 into the subclavian vein 16. The lead(s) 8 to be implanted are electrically coupled to the pulse generator 6 [block 110]. A PSA-programmer 18 is placed in communication with the pulse generator 6 [block 115]. For example, as indicated by arrow A in FIG. 3A, the communication between the PSA-programmer and the pulse generator may be wireless communication via a wand or programming head 20 electrically coupled to the PSA-programmer 18. SVC-to-Case vectors are generated between the case 22 of the pulse generator 6 and one or more lead SVC electrodes or coils 24 present in the SVC during implantation of the lead(s) in the patient's heart 26 [block 120]. ECI is assessed via the PSA-programmer during implantation of the lead(s) [block 125], the lead(s) being positioned to optimize the electrotherapy to the heart 26 based off the ECI assessment [block 130].

In some embodiments of the first method, other electrical vectors may be generated between the case 22 and the distal lead electrodes 27 or between the distal lead electrodes 27 and the SVC coil 24. These vectors may be analyzed during lead implantation to identify electrical characteristics to optimize lead placement for the optimization of electrotherapy to the heart. In one embodiment, the electrodes 27 may be the electrodes of a quadpolar (multi-polar) lead.

As can be understood from FIG. 3B, which is a diagram of a patient wherein another embodiment of the first method is employed, everything is generally the same as described above with respect to FIGS. 2 and 3A, except the lead(s) 8 are electrically coupled to a pod or electrical communication coupler 28 electrically coupled to the PSA-programmer 18, and a temporary electrical conductor 30 extends between the pulse generator and the coupler 28, thereby allowing the PSA-programmer to communicate back and forth.

In one embodiment, the pod 28 may be hardwired to the PSA-programmer 18 as indicated in FIG. 3B to allow communication between the pod and PSA-programmer. In another embodiment, the pod 28 and PSA-programmer 18 are configured for wireless communication with each other and, as a result, no hardwired arrangement exists between the two.

In one embodiment, as indicated in FIG. 3B, the pod 28 may be configured so that a stylet or guide wire 31 can extend through the pod 28 and into and through the lead 8 so as to allow the guidewire or stylet 31 to be used to manipulate the lead 8 into a desired implantation location.

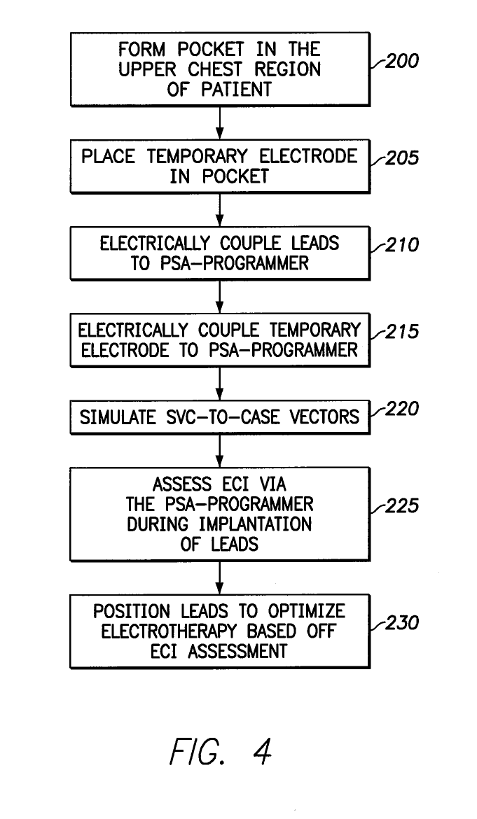

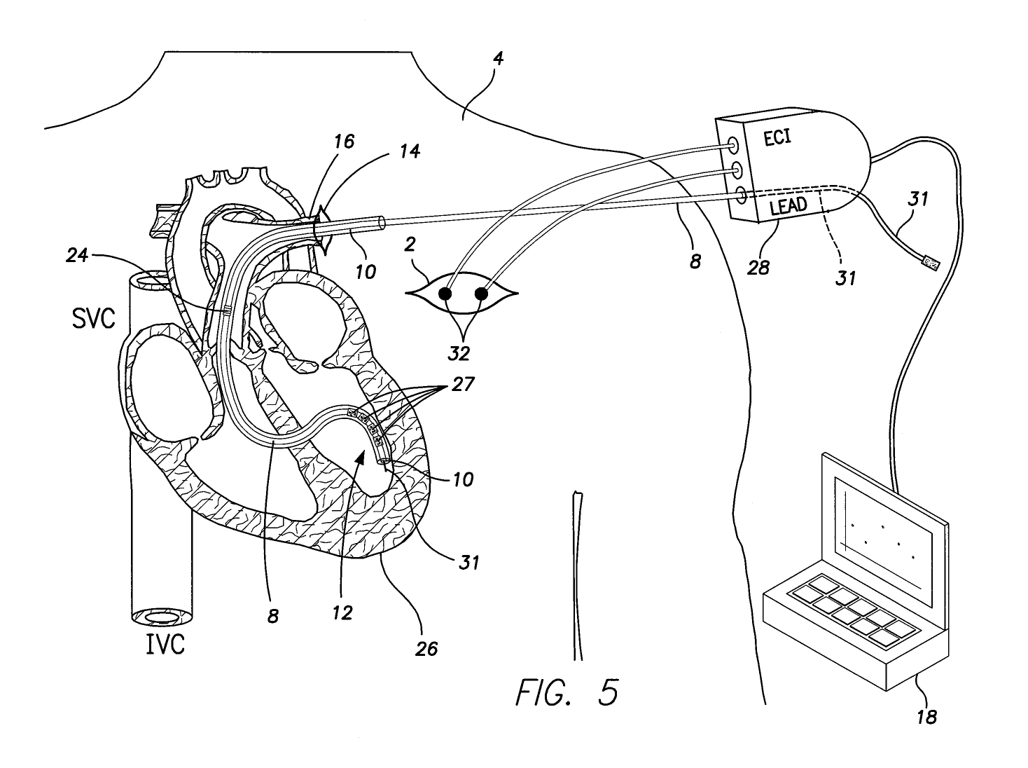

For a discussion of a second method of employing ECI analysis intra-operatively to optimize lead placement, reference is made to FIGS. 4 and 5, which are, respectively, a flow chart and a diagram of a patient wherein the second method is employed. As can be understood from FIGS. 4 and 5, temporary electrode(s) 32 are placed in the pocket 2 in place of the pulse generator 6 of the embodiment discussed with respect to FIGS. 2 and 3. For example, in one embodiment, a pocket 2 is formed in the upper chest region of the patient 4 [block 200]. The lead(s) 8 to be implanted extend into the patient's cardiovascular system via an introducer tubular body 10 extending to the implant location 12 through a percutaneous access 14 into the subclavian vein 16. The temporary electrode(s) 32 are placed in the pocket [block 205]. The lead(s) 8 to be implanted are electrically coupled to an electrical communication coupler 28 electrically coupled to a PSA-programmer 18 [block 210]. The temporary electrode(s) 32 are electrically coupled to the electrical communication coupler 28 electrically coupled to the PSA-programmer 18 [block 215]. SVC-to-Case vectors are simulated by generating vectors between the temporary electrode(s) 32 and one or more lead SVC electrodes or coils 24 present in the SVC during implantation of the lead(s) 8 [block 220]. ECI is assessed via the PSA-programmer 18 during implantation of the lead(s) 8 [block 225], the lead(s) being positioned to optimize the electrotherapy to the heart 26 based off the ECI assessment [block 230].

In some embodiments of the second method, other electrical vectors may be generated between the temporary electrodes 32 and the distal lead electrodes 27 or between the distal lead electrodes 27 and the SVC coil 24. These vectors may be analyzed during lead implantation to identify electrical characteristics to optimize lead placement for the optimization of electrotherapy to the heart.

Generally, physicians prefer to perform pocket construction after the leads have been implanted. Therefore, the first and second methods discussed above with respect to FIGS. 2-5 cannot be employed if ECI is to be analyzed during lead implantation to allow for lead implantation optimization. As a result, in a third embodiment, an electrode-equipped introducer tubular body 34 (e.g., catheter, sheath, etc.) is configured and employed as discussed below to replicate the electrical operational characteristics of a pulse generator case or temporary electrode located in a pocket formed in the patient.

In one embodiment, the pod 28 may be hardwired to the PSA-programmer 18 as indicated in FIG. 5 to allow communication between the pod and PSA-programmer. In another embodiment, the pod 28 and PSA-programmer 18 are configured for wireless communication with each other and, as a result, no hardwired arrangement exists between the two.

In one embodiment, as indicated in FIG. 5, the pod 28 may be configured so that a stylet or guide wire 31 can extend through the pod 28 and into and through the lead 8 so as to allow the guidewire or stylet 31 to be used to manipulate the lead 8 into a desired implantation location.

In one embodiment, the temporary electrode(s) 32 may be in the form of relatively small single or multiple point or snap electrodes as employed with EKG. Alternatively, the temporary electrode(s) 32 may be in the form of an electrode generally similar in size to the can of a pulse generator.

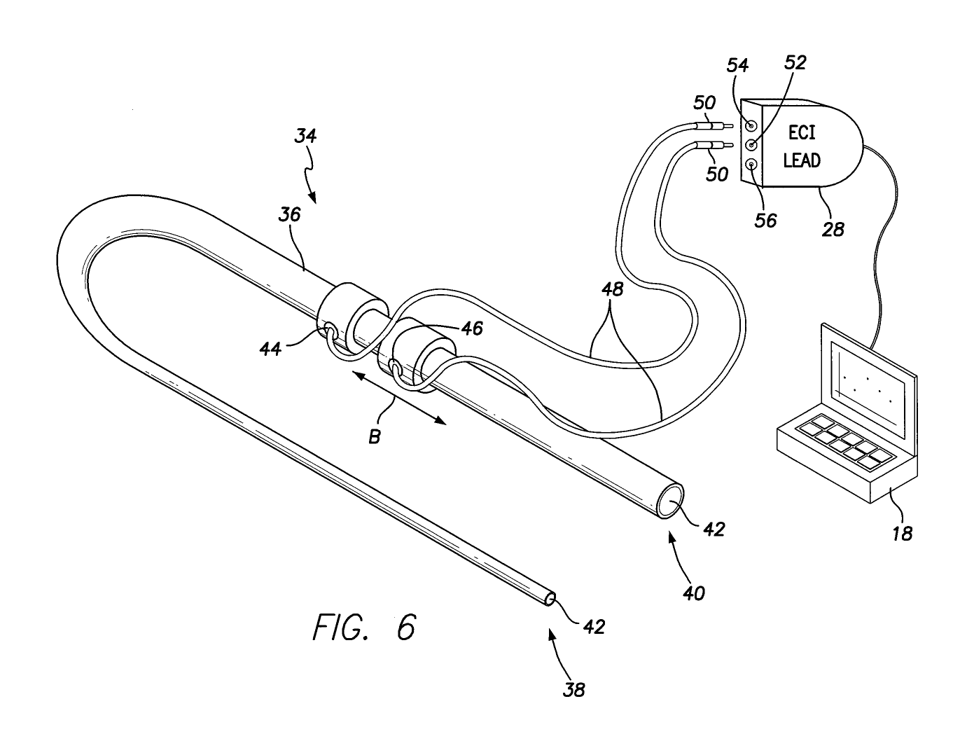

For a discussion of the features of one embodiment of the electrode-equipped introducer 34 configured for use in the third method, reference is made to FIG. 6, which is an isometric view of the introducer 34 and the PSA-programmer 18 for use therewith. As shown in FIG. 6, in one embodiment, the introducer 34 includes a tubular body 36 having a distal end 38, a proximal end 40 and a lumen 42 extending thought the tubular body. The tubular body may be flexible yet torqueable, may include radiopaque materials that facilitate the tubular body (or features thereof such as, for example, the distal end) to be visualized via fluoroscopy, and may be configured to bias into a desired shape such that the tubular body can be caused to enter a desired area of the patient's cardiovascular system. The distal end of the tubular body may be atraumatic. The introducer 34 further includes one or more electrodes 44, 46 supported on the tubular body 36 and electrical conductors 48 extending from the electrodes to terminate in electrical connectors 50 that are configured to be electrically coupled to corresponding ports 52, 54 in the electrical communication coupler 28, which is electrically coupled to a PSA-programmer 18. The coupler 28 also includes another port 56 configured to be electrically coupled to a lead connector end of the lead 8 to be implanted. As mentioned above with respect to FIGS. 3B and 5, the coupler 28 of FIG. 6 may also be configured to allow a guidewire or stylet 31 to extend through the coupler 28 and into and through the lead so as to allow the lead to be navigated into a desired implantation site.

In one embodiment, the pod 28 may be hardwired to the PSA-programmer 18 as indicated in FIG. 6 to allow communication between the pod and PSA-programmer. In another embodiment, the pod 28 and PSA-programmer 18 are configured for wireless communication with each other and, as a result, no hardwired arrangement exists between the two.

The electrodes 44, 46 are located near the proximal end 40 of the tubular body. As can be understood from arrow B in FIG. 6, one or more of the electrodes 44, 46 may be configured to be longitudinally displaceable along the tubular body 36.

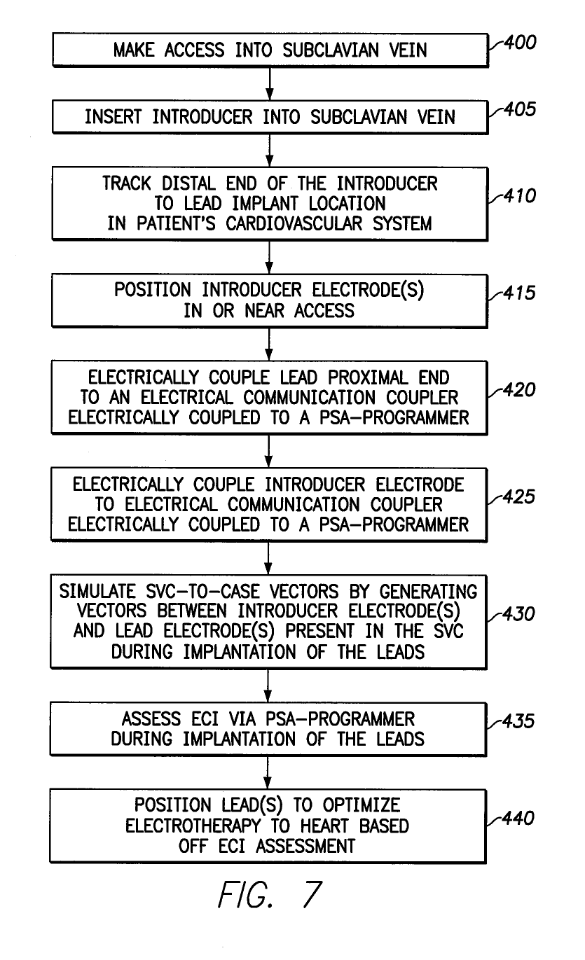

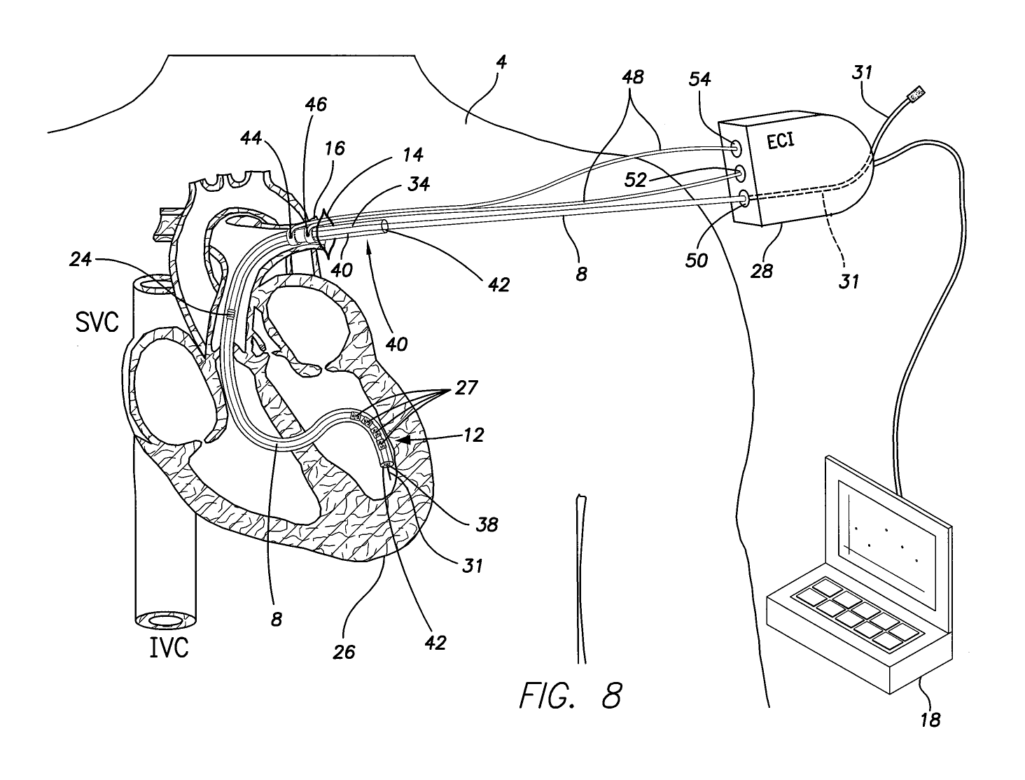

For a discussion of the third method of employing ECI analysis intra-operatively to optimize lead placement, reference is made to FIGS. 7 and 8, which are, respectively, a flow chart and a diagram of a patient wherein the third method is employed. As can be understood from FIGS. 7 and 8, the electrode-equipped introducer 34 is inserted into the patient 4, the electrode(s) 44, 46 on the introducer 34 being in the patient in the vicinity of the where the pocket 2 will eventually be formed in the patient 4 for the receipt of the pulse generator 6. Thus, the electrode(s) 44, 46 can be used to take the place of the pulse generator 6 of the embodiment discussed with respect to FIGS. 2 and 3 or the temporary electrode(s) 32 of the embodiment discussed with respect to FIGS. 4 and 5.

In one embodiment of the third method as indicated in FIGS. 7 and 8, an access 14 is made into the subclavian vein 16 of the patient 4 [block 400], and the introducer 34 is inserted via the access into the subclavian vein [block 405]. The distal end 38 of the introducer is tracked to the lead implant location 12 in the patient's cardiovascular system [block 410]. The introducer electrode(s) 44, 46, which are located on the introducer 34 near the proximal end 40 of the introducer, are positioned in or near the access 14 to generally replicate the location of the pulse generator 6 when implanted in a patient's body [block 415]. Because the electrodes 44, 46 are adjustably mounted on the tubular body 36, the location of the electrodes 44, 46 on the tubular body may be adjusted as needed to be positioned to replicate the pulse generator. A lead 8 is tracked through the lumen 42 of the introducer 34 to position the distal end of the lead 8, and more specifically, the SVC coil 24 in the SVC and the electrodes 27 near the distal end of the lead 8 near a desired lead implantation site 12 [block 415].

The proximal end of the lead 8 is electrically coupled to an electrical communication coupler 28 electrically coupled to a PSA-programmer 18 [block 420]. The introducer electrode(s) 44, 46 are electrically coupled to the electrical communication coupler 28 electrically coupled to the PSA-programmer 18 [block 425]. SVC-to-Case vectors are simulated by generating vectors between the introducer electrode(s) 44, 46 and one or more lead SVC coils 24 present in the SVC during implantation of the leads 8 [block 430]. ECI is assessed via the PSA-programmer during implantation of the leads [block 435], the leads being positioned to optimize the electrotherapy to the heart based off the ECI assessment [block 440].

As can be understood from FIG. 8, the coupler 28 may also be configured to allow a guidewire or stylet 31 to extend through the coupler 28 and into and through the lead so as to allow the lead to be navigated into a desired implantation site. Also, in one embodiment, the pod 28 may be hardwired to the PSA-programmer 18 as indicated in FIG. 8 to allow communication between the pod and PSA-programmer. In another embodiment, the pod 28 and PSA-programmer 18 are configured for wireless communication with each other and, as a result, no hardwired arrangement exists between the two.

In some embodiments of the third method, other electrical vectors may be generated between the introducer electrodes 44, 46 and the distal lead electrodes 27 or between the distal lead electrodes 27 and the SVC coil 24. These vectors may be analyzed during lead implantation to identify electrical characteristics to optimize lead placement for the optimization of electrotherapy to the heart.

While first and second methods may be employed to optimize lead implantation for the optimization of electrotherapy to the heart, some physicians may not like forming the pocket 2 prior to lead implantation. Accordingly, the third method addresses the issues with the first and second methods by providing an introducer 34 with electrodes 44, 46. The electrodes provide for a temporary impedance measurement between the SVC coil 24 of the lead 8 to be implanted and introducer electrodes. Hence the introducer electrodes are used as a surrogate for the case 22 of the pulse generator 6 prior to the pulse generator being implanted.

Because the electrodes are incorporated into a lead introducer, the electrodes can be used to provide an anode for pacing, an electrode to emulate the ICD case for common mode grounding of a cardiac electrogram (“EGM”) sensing system or for extra-cardiac impedance measurement. In one embodiment, the electrodes 44, 46 may be in the form of a snap electrode similar to the nipple-like metal electrodes found on an EKG electrode.

As can be understood from FIGS. 6 and 8, in one embodiment, the introducer 34 carries an extra electrode with a snap that locates under the skin of the patient near the future pocket that will receive the pulse generator. Since this electrode locates under the skin, this method is superior to a skin patch electrode. The extra electrode may allow the sheath to slip through the electrode to account for different physical body dimension.

In one embodiment, the pod or electrical communication coupler 28 electrically coupled to the programmer 18 will send a high frequency pulse necessary for ECI measurement. The programmer 18 will display peak-to-peak SVC-Case ECI as a surrogate on the screen to guide optimal placement.

Electrodes being supported on the introducer used in the third method allow for extra-cardiac impedance measurement within the patient while obviating the need for creating a pocket early during the implant surgery as is needed for the first and second methods. The introducer electrodes may alternatively be used to emulate a pulse generator case intraoperatively for any application. The introducer electrodes may also be used for electrogram sensing, as a unipolar pacing anode, or as a common mode electrode for electrogram sensing between implanted electrodes.

Although the present invention has been described with reference to preferred embodiments, persons skilled in the art will recognize that changes may be made in form and detail without departing from the spirit and scope of the invention.

What is claimed is:

1. A method of optimizing the implantation of an implantable medical lead into a patient to optimize electrotherapy administered via the lead, the method comprising:
inserting the lead into the patient, the lead including a first electrode;
providing a second electrode in the patient, wherein the second electrode is not part of the lead;
generating an electrical vector between the first electrode and second electrode, the electrical vector being generated as the lead is being implanted;
analyzing the electrical vector as the lead is being implanted; and
optimizing the implantation of the lead based off of the analysis of the electrical vector to optimize electrotherapy administered via the lead.

2. The method of claim 1, further comprising creating a pocket in an upper chest region of the patient and positioning the second electrode in the pocket.

3. The method of claim 2, wherein the second electrode is a case of a pulse generator positioned in the pocket.

4. The method of claim 2, wherein the second electrode is a temporary electrode positioned in the pocket.

5. The method of claim 4, further comprising removing the temporary electrode from the pocket once the lead is implanted as desired, positioning a pulse generator in the pocket and coupling a proximal end of the lead to the pulse generator.

6. The method of claim 1, further comprising providing a tubular body through which the lead is inserted into the patient and wherein the tubular body includes the second electrode.

7. The method of claim 6, wherein the second electrode is displaceable along the tubular body.

8. The method of claim 7, further comprising adjusting the second electrode along the tubular body to be maintained just within the patient as the tubular body is positionally adjusted within the patient during the course of implanting the lead.

9. The method of claim 8, further comprising removing the tubular body from the patient once the lead is implanted as desired, forming a pocket in the upper chest region of the patient, positioning a pulse generator in the pocket and coupling a proximal end of the lead to the pulse generator.

10. The method of claim 1, wherein the first electrode includes an SVC coil positioned in a SVC of the patient.

11. The method of claim 1, wherein the electrical vector at least simulates a SVC-to-Case vector.

12. The method of claim 1, wherein the electrical vector is analyzed with respect to extra-cardiac impedance.

13. The method of claim 1, wherein the electrical vector includes a constant current.

14. The method of claim 1, wherein analyzing the electrical vector as the lead is being implanted includes employing voltage as surrogate of impedance.

15. The method of claim 1, further comprising optimizing an AVD of the analysis of the electrical vector to optimize electrotherapy administered via the lead.

16. A method of employing extra-cardiac impedance to intra-operatively optimize lead placement, the method comprising:
during the implantation of a lead, generating an electrical vector between a lead electrode supported on the lead and another electrode not supported on the lead but at least partially positioned within a patient;
during the implantation of the lead, analyzing the electrical vector; and
guiding the implantation of the lead based at least in part off of the analysis of the electrical vector.

17. The method of claim 16, wherein the another electrode is at least one of a case of a pulse generator or a temporary electrode positioned within a pocket formed in an upper chest region of the patient.

18. The method of claim 16, wherein the another electrode is supported on a tubular body of an introducer catheter or sheath.

19. The method of claim 18, wherein the another electrode is positioned within a wound in the patient leading to the subclavian vein of the patient.

20. The method of claim 16, wherein the lead electrode includes an SVC coil positioned in a SVC of the patient.

21. The method of claim 16, wherein the electrical vector at least simulates a SVC-to-Case vector.

22. The method of claim 16, wherein the electrical vector is analyzed with respect to extra-cardiac impedance.

23. A delivery tool for implanting a lead into a patient, the tool comprising:
a tubular body comprising a proximal end, a distal end, and a lumen extending longitudinally through the tubular body between the proximal end and the distal end;
a first electrode supported on the tubular body near the proximal end in such a manner that the first electrode can be displaced longitudinally along the tubular body; and
a conductor extending proximally from the electrode.

24. The tool of claim 23, wherein the tubular body is part of an introducer sheath or a catheter.

25. The tool of claim 23, further comprising a system coupled to a proximal end of the conductor and configured to analyze an electrical vector generated between the first electrode and an electrode of a lead delivered via the tool.

26. The tool of claim 25, wherein the electrode of the lead is positioned in a SVC of a patient.

27. The tool of claim 25, wherein the system analyzes the electrical vector with respect to extra-cardiac impedance.

28. A method of optimizing an implantation of an implantable medical lead, the method comprising:
identifying a characteristic of extra cardiac impedance; and
employing the characteristic as a surrogate of cardiac output,
wherein the characteristic is monitored during lead implantation so as to position the lead to optimize cardiac output.

29. The method of claim 28, wherein the characteristic includes at least one of Zarea, slope, max, min, or peak-to-peak.

30. The method of claim 28, further comprising optimizing a pulse generator parameter to optimize cardiac output by monitoring the characteristic.

31. The method of claim 30, wherein the pulse generator parameter includes at least one of AVD, V-V timing, lead configuration, or pacing mode.
